(19) United States
(12) Patent Application Publication
(10) Pub. No.: US 2023/0377689 A1
(43) Pub. Date: Nov. 23, 2023

(54) SYSTEM AND METHOD OF ANTIBODY/ MACROMOLECULE DRUG AFFINITY MODIFICATION

(71) Applicant: Ainnocence Technologies LLC, MIAMI, FL (US)

(72) Inventors: Yue Kang, Shanghai (IN); Lurong Pan, Vestavia Hill, AL (US)

(21) Appl. No.: 17/811,091

(22) Filed: Jul. 7, 2022

(30) Foreign Application Priority Data
May 17, 2022 (CN) 2022105370156

(51) Int. Cl. G16B 35/10; C12N 15/10; G16B 30/00; G16B 45/00

(52) U.S. Cl. CPC G16B 35/10; C12N 15/1058; G16B 30/00; G16B 45/00

(57) ABSTRACT

The present invention provides an affinity modification system of antibody/macromolecular drug, wherein the affinity modification system comprises: an interaction module, set to: input template sequence information of antibody/macromolecular drugs, modification requirements of single/multi-targets of antibody/macromolecular drugs and optional user-defined screening requirements to generate interaction antibody/macromolecular drug sequence information; affinity modification module, set to: according to the interaction antibody/macromolecular drug sequence information, perform corresponding partial or exhaustive numeration of possible sequence in a part of the full variable range to obtain a mutation library, and perform sequence-based affinity prediction on the mutation library based on a deep learning model, so as to obtain the sequence information of the modified antibody/macromolecular drug; an output module, designed to: according to the sequence information of the modified antibody/macromolecular drug, output the sequence information of the candidate antibody/macromolecular drug. The invention also provides a corresponding affinity modification method.

### CROSS-REFERENCE TO RELATED APPLICATIONS

This application claims priority from a patent application filed in China having Patent Application No. 2022105370156 filed on May 17, 2022, and titled “A SYSTEM AND METHOD OF ANTIBODY/MACROMOLECULE DRUG AFFINITY MODIFICATION”.

### FIELD OF INVENTION

The invention relates to the field of artificial intelligence (AI)-assisted drug virtual screening and design, in particular to the field of affinity modification design of antibody drug/macromolecular drug.

### BACKGROUND

Affinity is a key parameter of antibody drugs, which usually affects the function and efficacy of antibodies. General speaking, antibodies or humanized antibodies produced by hybridoma cell technology already have relatively high affinity, but this affinity may not be sufficient to meet the needs of therapeutic antibody.

In the process of antibody/macromolecule design, the high affinity of the drug and the target molecule is a necessary condition for the drug to function. The increase of affinity is helpful to improve the specificity and efficacy of the antibody, while reducing the dosage and reducing the toxic and side effects.

Traditional affinity maturation techniques mainly include point mutation (site-directed mutation and random mutation), CDR region recombination, chain replacement, and DNA recombination. The traditional modification method has a high dependence on the antigen/target structure, so it is difficult to design the target of unknown structure or unknown epitope, and the screening hit rate is low.

However, due to the inherent limitations of these technologies in the display ability, it is still a challenge to construct a large-scale mutation library, which limits the ability to search the optimal sequence for a specific target in all mutation spaces of antibodies. In addition, the antibody selected in the experiment has the ability to bind to antigen, but it is still necessary for downstream experiments (such as Elisa) to further select the antibody with high binding force, so as to accurately evaluate the affinity strength between antibody and antigen. In addition, the traditional modification method is highly dependent on antigen/target structure, so it is difficult to design targets with unknown structure or unknown epitope, and the screening hit rate is low.

Finally, the traditional modification methods are generally only optimized for a specific target, so it is difficult to design drugs targeting multiple targets, and the design methods among different targets cannot be reused.

Based on the above, the present application provides a technical solution to solve the above technical problems.

### SUMMARY

The present invention provides an affinity modification system of antibody/macromolecular drugs directly optimized at the amino acid sequence level.

The present invention provides an affinity modification method of antibody/macromolecular drug directly optimized at amino acid sequence level.

The first aspect of the present invention provides an affinity modification system of antibody/macromolecular drug, wherein the affinity modification system comprises:
An interaction module, the interaction module is set to: input template sequence information of antibody/macromolecular drugs, modification requirements of single/multi-targets of antibody/macromolecular drugs and optional user-defined screening requirements to generate interaction antibody/macromolecular drug sequence information;
Affinity modification module, the affinity modification module is set to: according to the interaction antibody/macromolecular drug sequence information, perform corresponding partial or exhaustive numeration of possible sequence in a part of the full variable range to obtain a mutation library, and perform sequence-based affinity prediction on the mutation library based on a deep learning model, so as to obtain the sequence information of the modified antibody/macromolecular drug;
An output module, the output module is designed to: according to the sequence information of the modified antibody/macromolecular drug, output the sequence information of the candidate antibody/macromolecular drug.

In a preferred embodiment of the present invention, wherein,
in the affinity design module, the single quantity level of the mutation library is not less than 10¹⁰.

In a preferred embodiment of the present invention, wherein,
in the affinity design module, the variable range includes one or more variable regions, variable spaces, variable number of sites or combinations thereof.

In a preferred embodiment of the present invention, wherein,
in the interaction module, the template sequence information of the antibody/macromolecular drug includes antigen/antibody template sequence, protein/protein template sequence, or protein/polypeptide template sequence of the antibody/macromolecular drug.

In a preferred embodiment of the present invention, wherein,
in the interaction module, in the modification requirements of single/multiple targets of the antibody/macromolecular drug.

Marking or specifying the variable range; and/or
Define the modification direction.

In a preferred embodiment of the present invention, wherein, the output module further comprises a visual analysis display module.

In a preferred embodiment of the present invention, wherein, the visual analysis display module provides the complete sequence information of the sequence information of the candidate antibody/macromolecular drug.

In a preferred embodiment of the present invention, wherein, the visual analysis display module further comprises a comparative analysis of the template sequence information of the antibody/macromolecular drug and the sequence information of the candidate antibody/macromolecular drug in a variable range.

The second aspect of the present invention provides an antibody/macromolecular drug affinity modification method, wherein the method comprises:
input template sequence information of antibody/macromolecular drugs, modification requirements of single/multi-targets of antibody/macromolecular drugs and optional user-defined screening requirements to generate interaction antibody/macromolecular drug sequence information;
according to the interaction antibody/macromolecular drug sequence information, perform partial or exhaustive numeration of possible sequence in a part of the full in a variable range to obtain a mutation library, and perform sequence-based affinity prediction on the mutation library based on a deep learning model, so as to obtain the sequence information of the modified antibody/macromolecular drug;
according to the sequence information of the modified antibody/macromolecular drug, output the sequence information of the candidate antibody/macromolecular drug.

In a preferred embodiment of the present invention, wherein, when perform partial or exhaustive numeration of possible sequence in a part of the full, the single quantity level of the mutation library is not less than 10¹⁰.

The present invention can bring at least one of the following beneficial effects:
screen the amino acid sequence of antibody/fusion protein up to one billion-level mutation spaces, significantly improve the screening hit rate of high affinity antibodies/macromolecules, and greatly reduce the time and screening cost of downstream experiments. In addition, the invention does not depend on the structural information or epitope information of antigen/target, and can directly optimize the virtual affinity maturation of antibody/macromolecule from the amino acid sequence level, which plays an important auxiliary role in macromolecular drug design of new target. More importantly, the virtual affinity module of the invention adopts a fully automatic calculation process, has a fast screening speed (the screening of one billion-level mutation spaces takes hours as a unit), and can simultaneously screen for multiple affinity modification conditions of multiple targets.

### BRIEF DESCRIPTION OF THE DRAWINGS

The preferred embodiments will be described below in a clear and easy-to-understand manner with reference to the drawings, and the above-mentioned characteristics, technical features, advantages and implementations thereof will be further described.

FIG. 1 is an example of the structural diagram of the automatically virtual antibody/protein affinity modification module of the present invention;

FIG. 2 is an example of the flowchart of the automatically virtual antibody/protein affinity modification of the present invention.

### DETAILED DESCRIPTION OF THE DISCLOSURE

Various aspects of the invention are described in further detail below.

Unless otherwise defined or described all professional and scientific terms used herein have the same meanings as those familiar to persons skilled in the art. In addition, any method and material similar or equivalent to those described can be used in the methods of the present invention.

Terminology is explained below.

For purposes herein, unless otherwise indicated, “/” is represented in an “and/or” relationship.

For example, “antibody/macromolecular drug” described in the present invention means that the affinity modification object may include antibodies and/or macromolecular drugs. The meanings of the antibodies and macromolecular drugs are known to persons skilled in the art.

For example, the “/” of “antigen/antibody template sequence, protein/protein template sequence, or protein/polypeptide template sequence” describe in the present invention represents the relationship of “and/or”.

Unless otherwise clearly specified and limited, “or” described in the present invention includes the relationship of “and”. The “and” is equivalent to the Boolean logical operator “AND”, the “or” is equivalent to the Boolean logical operator “OR”, and “AND” is a subset of “OR”.

It will be understood that, although the terms“first,” “second,” etc. may be used herein to describe different elements, but these elements should not be limited by these terms. These terms are only used to distinguish one element from another. Therefore, a first element could be called a second element without departing from the teachings of the present inventive concept.

In the present invention, the term “containing”, “including”, or “comprising” means that various ingredients may be employed together in the mixture or composition of the present invention. Therefore, the terms “consisting essentially of” and “consisting of” are included in the term “containing”, “including”, or “comprising”.

Unless otherwise clearly specified and limited, terms “link”. “connect”, and “connection” should be understood in a broad sense. For example, the terms may be used for a fixed connection, a connection through intermediate media, an internal connection between two elements, or an interaction relationship between two elements. Persons of ordinary skill in the art may understand specific meanings of the terms in the embodiments of this application based on specific cases.

For example, if an element (or component) is called to be on, coupled with or connected to another element, then the one element may be directly formed on, coupled with or connected to the other element, or there may be one or more intervening elements between them. On the contrary, if the expressions “directly on ⋅ ⋅ ⋅ ”, “directly coupled with ⋅ ⋅ ⋅ ” and “directly connected with ⋅ ⋅ ⋅ ” are used here, it means that there are no intervening elements. Other words used to explain the relationship between elements should be similarly interpreted, such as “between ⋅ ⋅ ⋅ ” and “directly between ⋅ ⋅ ⋅ ”, “attached” and “directly attached”, “adjacent” and “directly adjacent”, and so on.

In addition, the words “front”, “back”, “left”, “right”, “up” and “down” used in the following description refer to directions in the drawings. The terms “inside” and “outside” used in refer to the direction towards or away from the geometric center of a specific part, respectively. It is understood that these terms are used here to describe the relationship of one element, layer or region with respect to another element, layer or region as shown in the drawings. In addition to the orientations described in the drawings, these terms should also include other orientations of devices.

Other aspects of the invention will be obvious to persons skilled in the art due to the disclosure herein.

In order to explain the embodiments of the present invention or the technical solutions more clearly in the prior art, the embodiments of the present invention will be described below with reference to the drawings. Obviously, the drawings in the following description are only some embodiments of the present invention. For persons of ordinary skill in the art, other drawings and other embodiments can be obtained according to these drawings without any creative effort.

It should also be noted that the illustrations provided in the following examples illustrate the basic concepts of the present application by way of illustration only. The drawings only show the components related to the present application and are not drawn according to the number, shape and size of the components in the actual implementation. The type, number and proportion of each component in the actual implementation may be changed at will, and the layout type of the components may be more complicated. For example, the thickness of elements in the drawings may be exaggerated for clarity.

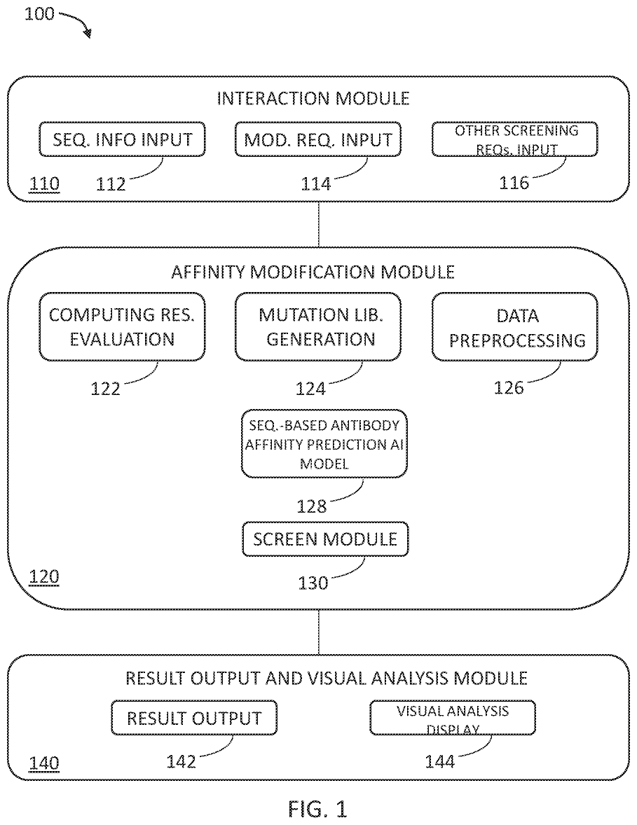

FIG. 1 is an example of the structural diagram 100 of the automatically virtual antibody/protein affinity modification module 120 of the present invention.

In the current single/multi-target affinity modification module 120 of antibody drugs or macromolecular drugs, the following am several common scenarios that lead to new problems, and the corresponding solutions adopted to solve the new problems:
In the aspects of computational-assisted affinity engineering, the traditional computer simulation methods calculate the antibody-antigen binding strengths according to the atomic chemical properties such as polarity and charge [see Reference 1]. With the rapid development of machine learning and deep learning research fields, AI-based algorithms and techniques have been explored for application in antibody development [see Reference 2]. Then, the prediction of interacting contacts was then investigated, and binding predictions were made by using surface-based geometric features: and then a topology-based network tree was employed to predict binding affinity changes based on the 3D structure of the complex [see Reference 3]. A long-term and short-term memory model 128 for antigen-specific affinity prediction was then trained on an in computer sequence library [Reference 4]. Thereafter, the mutations in CDR-H3 were used for sequence-based deep learning antibody design for computer antibody affinity maturation [Reference 5].

The references are as follows:
[Reference 1]
Xue. Li C., et al., “PRODIGY: a web server for predicting the binding affinity of protein-protein complexes”, [symbol] Bioinformatics [symbol] 32.23 (2016): 3676-3678.
[Reference 2]
Gainza. Pablo, et al., “Deciphering interaction fingerprints from protein molecular surfaces using geometric deep learning”, [symbol] Nature Methods [symbol] 17.2 (2020): 184-192.
[Reference 3]
Wang, Menglun, Zixuan Cang, and Guo-Wei Wei, “A topology-based network tree for the prediction of protein-protein binding affinity changes following mutation”, [symbol] Nature Machine Intelligence [symbol] 2.2 (2020): 116-123.
[Reference 4]
Mason, Derek M. et al.: “Deep learning enables therapeutic antibody optimization in mammalian cells by deciphering high-dimensional protein sequence space”, [symbol] BioRxiv [symbol] (2019): 617860.
[Reference 5]
Liu Ge, et al., “Antibody complementarity determining region design using high-capacity machine learning”. [symbol] Bioinformatics [symbol] 36.7 (2020): 2126-2133.

To sum up, the above computational-based antibody affinity prediction methods either rely on the 3D structural information of antibodies and antigens, or rely on artificially defined chemical characteristics, or rely on the determined epitope information, which limits the application of the above tools to unknown structural targets.

In addition, the verification manners of the affinity model of calculation and screen, using a screen module 130, above are mainly divided into two categories, one is to train and test the backtracking data 126 of a single target, and the other is to include the known target data 126 in the training data 126 in the test data 126. The above two methods are difficult to directly reflect the generalization ability of the model in other antigen-antibody affinities, which limits the practical application in pharmaceutical process.

In addition, both traditional experimental and computational-assisted cannot avoid the limited space for antibody modification, the modification methods partially or completely depend on the antigen/target structural information. The experimental construction or model construction aimed at one or a certain type of target, and the time cost high, the cost of downstream experiments is high, and the design methods are not universal etc.

In view of the above problems, the purpose of the present invention is to overcome the following shortcomings: the traditional antibody affinity maturation technology adopts random mutation or computer-assisted site-directed mutation (such as point mutation only for CDR-H3 region of antibody) to generate antibody mutation library 124, which has high experimental construction cost and long experimental period. At the same time, limited by the experimental cost and calculation methods, the above methods have limited imagination space and high randomness for molecular modification, and it is difficult to directly confirm the improvement degree of affinity through screening, using the screen module 130, so that the cost of verifying affinity in downstream experiments is higher.

In view of the above shortcomings, the invention aims to solve the limitations of traditional artificial design methods and traditional computer-aided methods, screen the amino acid sequence of antibody/fusion protein up to one billion-level mutation spaces, significantly improve the screening hit rate of high affinity antibodies/macromolecules, and greatly reduce the time and screening cost of downstream experiments. In addition, the invention does not depend on the structural information or epitope information of antigen/target, and can directly optimize the virtual affinity maturation of antibody/macromolecule from the amino acid sequence level, which plays an important auxiliary role in macromolecular drug design of new target. More importantly, the virtual affinity module of the invention adopts a fully automatic calculation process, has a fast screening speed (the screening of one billion-level mutation spaces takes hours as a unit), and can simultaneously screen for multiple affinity modification conditions of multiple targets.

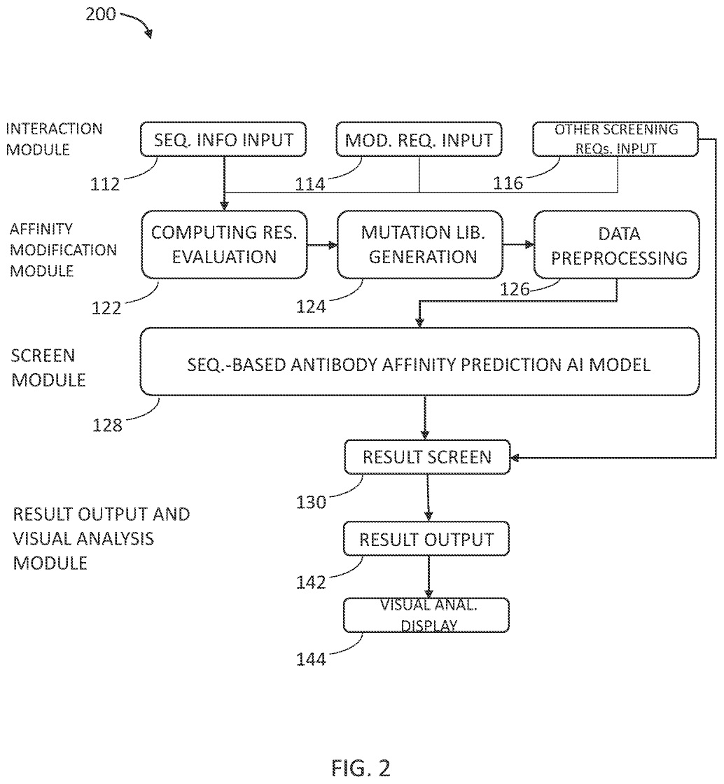

FIG. 2 is an example of the flowchart 200 of the automatically virtual antibody/protein affinity modification of the present invention.

The embodiments of the present invention and the technical progress are described by way of examples below.

The first aspect of the present invention provides an antibody/macromolecular drug single/multi-target affinity modification system, wherein the affinity modification system comprises:
An interaction module 110, the interaction module 110 is set to: input template sequence information 112 of antibody/macromolecular drugs, modification requirements of single/multi-targets of antibody/macromolecular drugs and optional user-defined screening requirements 116 to generate interaction antibody/macromolecular drug sequence information 112;
Affinity modification module 120, the affinity modification module 120 is set to: according to the interaction antibody/macromolecular drug sequence information 112, perform partial or exhaustive numeration of possible sequence in a part of the full variable range to obtain a mutation library 124, and perform sequence-based affinity prediction on the mutation library 124 based on a deep learning model, so as to obtain the sequence information 112 of the modified antibody/macromolecular drug;
An output module 140, the output module 140 is designed to: according to the sequence information 112 of the modified antibody/macromolecular drug, output the sequence information 112 of the candidate antibody/macromolecular drug.

In a preferred embodiment of the present invention, in the affinity design module, the single quantity level of the mutation library 124 is not less than 10¹⁰.

In a preferred embodiment of the present invention, in the affinity design module, the variable range includes one or more variable regions, variable spaces, variable number of sites or combinations thereof.

In a preferred embodiment of the present invention, in the interaction module 110, the template sequence information 112 of the antibody/macromolecular drug includes antigen/antibody template sequence, protein/protein template sequence, or protein/polypeptide template sequence of the antibody/macromolecular drug.

In a preferred embodiment of the present invention, in the interaction module 110, in the modification requirements of single/multiple targets of the antibody/macromolecular drug,
Marking or specifying the variable range; and/or

Define the modification direction.

In a preferred embodiment of the present invention, wherein the output module 140 further comprises a visual analysis display module 144.

In a preferred embodiment of the present invention, the visual analysis display module 144 provides the complete sequence information 112 of the sequence information 112 of the candidate antibody/macromolecular drug.

In a preferred embodiment of the present invention, the visual analysis display module 144 further comprises a comparative analysis of the template sequence information 112 of the antibody/macromolecular drug and the sequence information 112 of the candidate antibody/macromolecular drug in a variable range.

In a specific embodiment of the present invention, an automatic virtual antibody/macromolecule affinity maturation technology based on data 126-driven and artificial intelligence algorithm is provided.

The invention includes: an affinity maturation interaction module 110, an affinity maturation design module based on artificial intelligence, and an affinity maturation visual analysis display module 144. The interaction module 110 requires the user to input antigen/antibody template sequence (or protein/protein, protein/polypeptide), wherein, the antigen/target can be multiple sequences. This module allows users to mark and specify the variable region (variable region) and the variable space range of interest, and define the modification direction of a single target one by one (affinity enhancement or weakening). It also allows to define the number of antibody sequences produced by virtual screening according to the use's situation (such as the estimated cost of the downstream experiment).

Sequence information 112 (and other user-defined information) is input from the interaction module 110 to the calculation module, according to the upstream information, the affinity maturation design module exhausts the variable space range of antibodies to generate antibody mutation library 124. The single mutation library 124 level can reach 10¹⁰. The calculation module preprocesses the sequence information 112 in the library one by one, and calculates and records the affinity of antibody antigen based on the deep learning model. Finally, the qualified antibody sequences are screened and output according to the user-defined screening conditions.

All antibody/protein candidate modified sequences generated by the design module enter the visual analysis display module 144. The visualization module provides mutation sites comparison of template sequences and candidate modification sequences, statistical charts of mutation sites, and the display of mutation sites thermal map, etc.

More specifically, as shown in FIGS. 1 and 2, in one embodiment, the affinity modification module 120 based on artificial intelligence of the present invention includes an affinity modification interaction module 110, an affinity modification design module based on artificial intelligence, and a result output 142 and visual analysis display module 144. The target users of the invention are the biological drugs/antibody drugs researchers.

The design/operation steps of affinity modification module 120 are as follows:
S1. The interaction module 110 is the user input interface, allowing the user to input antigen sequence, antibody sequence (or target protein/drug protein sequence). Among them, the antigen/target can be multiple sequences, and the modification direction of a single target can be defined one by one (affinity enhancement or weakening). This module allows users to mark and specify the variable region (variable region) and variable space range of interest, define the modification direction (affinity enhancement or weakening), and define the number of antibody sequences produced by virtual screening according to the user's situation (such as the estimated cost of the downstream experiment). For example, to optimize the antibody template of a certain antigen, it is necessary to fill in the complete sequence information 112 of the antibody antigen, and fill in the modification requirements of antibody affinity, that is, to enhancement or weakening. At the same time, users can choose to limit the mutation site to a certain position range, such as the CDR-H3 region of the antibody. The input module allows users to customize multiple regions of interest. At the same time, users can also define the number of mutation sites, and can choose single-point mutation, double-point mutation or multi-point mutation (3-5 points). Finally, the user can define the number of candidate antibody sequences given by the module according to the actual situation (such as the estimated cost of the downstream experiment).
S2, the calculation module receives the amino acid sequence information 112, the modification direction information and other user-defined information provided by the interaction module 110. According to the upstream information, the affinity maturation design module evaluates the mutation space of the antibody. If the mutation space exceeds the calculated maximum upper limit of 10¹⁰, it will prompt to narrow the mutation range or adopt the mutation range recommended by the module for screening. In the calculation process, the calculation module preprocesses the candidate mutation amino acid sequences one by one, and calculates and records the affinity of antibody antigens one by one based on the deep learning model. After the calculation is completed, the module scores and orders all candidate antibody sequences, and the N sequences with the highest affinity (the modification direction is enhanced) or the lowest affinity (the modification direction is weakened) will be the final modification sequence, wherein N is the number of user-defined sequences, and the default output sequence number is 200.
S3, the visual analysis display module 144 accepts all antibody/protein candidate modification sequences generated by the design module. The visual analysis display module 144 provides the information of the complete antibody sequence, and at the same time, provides the mutation sites comparison between template sequences and candidate modified sequences, and statistical chart of mutation sites, such as mutation sites contained in CDRH1, H2 and H3 regions of the antibody respectively. In addition, the display of mutation sites thermal map is provided, including the original amino acid type of each mutation site and the amino acid type after mutation. In addition, the species of the mutated amino acids are also displayed. Classification and group mainly considers the physical and chemical properties of amino acids, and is divided into five groups: polar, nonpolar, aromatic, positively charged and negatively charged.

To sum up, the specific embodiments of the present invention as shown in FIGS. 1 and 2 prove that the following effects are indeed achieved:
The invention can solve the limitations of the traditional manual design method and the traditional computer-assisted method, obviously improve the screening hit rate of high affinity antibodies, and greatly reduce the time and screening cost of downstream experiments.

Specifically, the present invention does not depend on the structural information or antigen epitope information of antibody antigen, and can directly optimize the virtual affinity maturation of the antibody from the amino acid sequence level of the antigen antibody, and achieves a high hit rate, thus providing a feasible solution for the design of biological drugs/antibody drugs in new targets of unknown institutions or targets with uncertain epitopes.

In addition, relying on the algorithm design and efficient computing resource allocation method, the present invention can search the mutation space of antibody/protein 10¹⁰ in a single time, breaking through the imagination barrier and calculation barrier in the traditional design, and allowing users to find the optimal solution for specific antigen in the super-large mutation space, so as to improve the hit rate and strength of affinity maturity.

More importantly, the virtual affinity module of the present invention adopts a fully automatic calculation process, and the calculation process and calculation method are not limited to one target or a certain kind of target. In addition, the virtual screening speed of the module is greatly increased (the screening of one billion-level mutation spaces takes hours as a unit), and multiple affinity modification conditions of multiple targets can be simultaneously screened, which has important auxiliary significance for new drug and multi-target drug research and development.

Based on the present application, persons skilled in the art should understand that an aspect described herein can be implemented independently from any other aspects, and two or more of these aspects can be combined in various ways. For example, any number of aspects set forth herein can be used to implement devices and/or to practice methods. In addition, other structures and/or functionalities other than one or more of the aspects set forth herein may be used to implement the device and/or to practice the method.

Persons skilled in the art shall understand that, in addition to implementing the system provided by the present invention and its various devices, modules, and units in a pure computer-readable program code manner, they may also be implemented to realize the same functions in the form of logic gates, switches, application-specific integrated circuits, programmable logic controllers, embedded microcontrollers, and the like, by performing logic programming on the steps of the method. Therefore, the system provided by the present invention and its various devices, modules and units can be regarded as hardware components, and the devices, modules, and units included in the system for implementing various functions can also be regarded as structures within the hardware components. Besides, the devices, modules, and units for realizing various functions can also be regarded as not only software modules but also the structures within the hardware components for implementing the method.

It should be noted that the above examples can be freely combined as required. The above are merely preferred embodiments of the present invention. It should be pointed out that for those of ordinary skill in the art, several improvements and modifications can be made without departing from the principle of the present invention, and these improvements and modifications should be regarded as the protection scope of the present invention.

All documents mentioned in the present invention are incorporated herein by reference, as if each one is individually incorporated by reference. Additionally, it should be understood that after reading the above teachings, persons skilled in the art can make various changes and modifications to the present invention. These equivalents also fall within the scope defined by the appended claims.

1. An affinity modification system of antibody/macromolecular drug, wherein the affinity modification system comprises:
an interaction module, the interaction module is set to: input template sequence information of antibody/macromolecular drugs, modification requirements of single/multi-targets of antibody/macromolecular drugs and optional user-defined screening requirements to generate interaction antibody/macromolecular drug sequence information;
an affinity modification module, the affinity modification module is set to: according to the interaction antibody/macromolecular drug sequence information, perform corresponding partial or exhaustive numeration of possible sequence in a part of the full variable range to obtain a mutation library, and perform sequence-based affinity prediction on the mutation library based on a deep learning model, so as to obtain the sequence information of the modified antibody/macromolecular drug; and
an output module, the output module is designed to: according to the sequence information of the modified antibody/macromolecular drug, output the sequence information of the candidate antibody/macromolecular drug.

2. The affinity modification system of antibody/macromolecular drug according to claim 1, wherein,
in the affinity design module, a single quantity level of the mutation library is not less than 10¹⁰.

3. The affinity modification system of antibody/macromolecular drug according to claim 1, wherein,
in the affinity design module, the variable range includes one or more variable regions, variable spaces, variable number of sites, or combinations thereof.

4. The affinity modification system of antibody/macromolecular drug according to claim 1, wherein,
in the interaction module, the template sequence information of the antibody/macromolecular drug includes at least one element of a set comprising an antigen/antibody template sequence, a protein/protein template sequence, and a protein/polypeptide template sequence of the antibody/macromolecular drug.

5. The affinity modification system of antibody/macromolecular drug according to claim 1, wherein,
in the interaction module, in the modification requirements of single/multiple targets of the antibody/macromolecular drug, further comprising:
at least one element of a set comprising marking the variable range and specifying the variable range; and
defining a modification direction.

6. The affinity modification system of antibody/macromolecular drug according to any one of claim 1-5, wherein, the output module further comprises a visual analysis display module.

7. The affinity modification system of antibody/macromolecular drug according to claim 6, wherein, the visual analysis display module provides the complete sequence information of the sequence information of the candidate antibody/macromolecular drug.

8. The affinity modification system of antibody/macromolecular drug according to claim 7, wherein, the visual analysis display module further comprises a comparative analysis of the template sequence information of the antibody/macromolecular drug and the sequence information of the candidate antibody/macromolecular drug in a variable range.

9. An affinity modification method of antibody/macromolecular drug, wherein the method comprises:
an input template sequence information of antibody/macromolecular drugs, modification requirements of single/multi-targets of antibody/macromolecular drugs, and optional user-defined screening requirements to generate interaction antibody/macromolecular drug sequence information;
according to the interaction antibody/macromolecular drug sequence information, perform partial or exhaustive numeration of possible sequence in a part of the full variable range to obtain a mutation library, and perform a sequence-based affinity prediction on the mutation library based on a deep learning model, so as to obtain the sequence information of the modified antibody/macromolecular drug; and
according to the sequence information of the modified antibody/macromolecular drug, output the sequence information of the candidate antibody/macromolecular drug.

10. The affinity modification method according to claim 9, wherein, when performing the partial or exhaustive numeration of possible sequence in a part of the full, a single quantity level of the mutation library is not less than 10¹⁰.
